(19) United States
(12) Patent Application Publication
(10) Pub. No.: US 2023/0051189 A1
(43) Pub. Date: Feb. 16, 2023

(54) COSMETIC PROCESS USING MICRONEEDLE SHEET

(71) Applicant: L'Oreal, Paris (FR)

(72) Inventors: Shu LIU, Kawasaki-shi, Kanagawa (JP); Gaurav AGARWAL, Kawasaki-shi, Kanagawa (JP)

(73) Assignee: L'Oreal, Paris (FR)

(21) Appl. No.: 17/785,233

(22) PCT Filed: Dec. 4, 2020
(86) PCT No.: PCT/JP2020/046186

§ 371 Date: Jun. 14, 2022

(30) Foreign Application Priority Data
Dec. 16, 2019 (JP) 2019-226590

(51) Int. Cl. A61M 37/00

(52) U.S. Cl. CPC A61M 37/0015; A61M 37/0076; A61M 2210/04; A61M 2210/0625

(57) ABSTRACT

The present invention relates to a cosmetic process for a keratin substance such as the skin or the lips, comprising the steps of: applying onto the keratin substance at least one composition comprising at least one coloring agent; and applying onto the keratin substance, to which the composition has been applied, a microneedle sheet comprising a substrate sheet and a plurality of microneedles on the substrate sheet such that the microneedles are capable of penetrating into the keratin substance. According to the present invention, microneedles can penetrate into a keratin substance to provide the keratin substance with long lasting and uniform color effects.

### TECHNICAL FIELD

The present invention relates to the use of a microneedle sheet comprising a plurality of microneedles for cosmetic treatments of a keratin substance such as skin and lips.

### BACKGROUND ART

A topical application of coloring agents such as dyes or pigments on skin by applying a formulation including the coloring agents is well known. For example, the surface of skin or lips is often colored or dyed with make-up cosmetic products such as a foundation and a lip stick. These make-up cosmetic products are widely used to color or dye skin, in particular face skin, and lips.

However, coloring or dyeing with make-up cosmetic products is temporary, and can be easily removed from the skin and lips by, for example, sebum, sweat and rain. Thus, users of make-up cosmetic products need to color skin and/or lips on a daily basis. Therefore, there is a need to provide long lasting coloring effects.

One of possible options to achieve long lasting coloring effects is to deliver coloring agents inside skin or lips. However, topical application of a formulation including coloring agents cannot provide sufficient penetration of the coloring agents into skin or lips. A thin layer of stratum corneum of the skin or lips is a major barrier for substances to penetrate into skin or lips. Therefore, the most important point for delivering coloring agents inside skin or lips is to pass through the stratum conium. Also, uniform dispersion of the coloring agents inside skin or lips is also important.

### DISCLOSURE OF INVENTION

An objective of the present invention is to provide a cosmetic process which can provide a keratin substance such as skin and lips with long lasting and uniform coloring effects.

The above objective can be achieved by a cosmetic process for a keratin substance such as skin and lips, comprising the steps of:

applying onto the keratin substance at least one composition comprising at least one coloring agent; and

applying onto the keratin substance, to which the composition has been applied, a microneedle sheet comprising a substrate sheet and a plurality of microneedles on the substrate sheet such that the microneedles are capable of penetrating into the keratin substance.

The microneedle may have a height of from 10 to 500 microns, preferably from 30 to 300 microns, and more preferably from 50 to 500 microns.

The microneedle may be in the shape of a pyramid.

The base of the microneedle may have a width of from 10 to 500 microns, preferably from 10 to 300 microns, and more preferably from 10 to 100 microns.

The ratio of (the height of the pyramid)/(the width of the base of the pyramid) of the microneedle may be 1 or more, preferably 1.5 or more, and more preferably 2.0 or more.

The microneedles may be made from inorganic materials, preferably metals or non-metals, and more preferably silicon.

The microneedles may be capable of penetrating into the keratin substrate with repetitions. The repetitions may be based on the vibrating or tapping of the microneedle sheet.

The coloring agent may be selected from dyes, pigments and mixtures thereof.

The amount of the coloring agent may be from 0.001% to 20% by weight, preferably from 0.01% to 15% by weight, and more preferably from 0.1% to 10% by weight, relative to the total weight of the composition.

The microneedle sheet may be applied onto the keratin substance without a step of drying the composition on the keratin substance.

The present invention also relates to a kit for coloring a keratin substance, such as skin and lips, comprising:

a microneedle sheet comprising a substrate sheet and a plurality of microneedles on the substrate sheet;

a device for applying the microneedle sheet onto the keratin substance such that the microneedles are capable of penetrating into the keratin substance; and a composition comprising at least one coloring agent.

The device in the above kit may be capable of applying the microneedle sheet with repetitions.

The device in the above kit may comprise at least one element which is capable of vibrating or tapping the microneedle sheet.

The present invention also relates to a use of at least one composition comprising at least one coloring agent in combination with a microneedle sheet comprising a substrate sheet and a plurality of microneedles on the substrate sheet, in order to provide a keratin substance such as skin or lips with long-lasting and uniform color.

### BRIEF DESCRIPTION OF DRAWINGS

FIG. 1 shows a schematic view of a device which is preferably used for the present invention.

FIG. 2 shows examples regarding how to use the present invention for lips.

FIG. 3 shows examples regarding how to use the present invention for around eyes.

FIG. 4 shows photographs of the surfaces of the skins used in Example 1 and Control.

FIG. 5 shows photographs of the cross sections of the skins used in Example 2 and Control.

### BEST MODE FOR CARRYING OUT THE INVENTION

After diligent research, the inventors have discovered that it is possible to provide a cosmetic process which can provide a keratin substance such as skin and lips with long lasting and uniform coloring effects.

Thus, one aspect of the present invention is a cosmetic process for a keratin substance such as skin and lips, comprising the steps of:

applying onto the keratin substance at least one composition comprising at least one coloring agent; and

applying onto the keratin substance, to which the above composition has been applied, a microneedle sheet comprising a substrate sheet and a plurality of microneedles on the substrate sheet such that the microneedles are capable of penetrating into the keratin substance.

According to the present invention, microneedles can penetrate into a keratin substance to provide the keratin substance with long lasting and uniform color effects.

Specifically, the microneedles can puncture the surface of skin or lips to create fine holes or passages through which the coloring agent can go into the skin or lips. Thus, the coloring agent can penetrate into the skin or lips through the fine holes or passages such that it can go into the stratum corneum and/or it may reach the bottom of the stratum corneum.

Furthermore, the coloring agent may pass through the stratum corneum to reach any one of the lower layers of the epidermis, such as a granular layer, a stratum spinosum and a basal layer. Therefore, the coloring agent can remain in skin or lips for a long period of time. This can provide long lasting coloring effects.

Also, the coloring agent which passes through the stratum corneum can relatively easily diffuse on or in the epidermis to uniformly distribute in the skin or lips. Therefore, this can provide uniform coloring effects.

Since microneedles do not cause any pain, the cosmetic process according to the present invention can provide cosmetic treatments without pain.

In addition, the cosmetic process according to the present invention can be performed in a relatively short period of time.

Thus, the present invention is convenient for cosmetic or non-therapeutic treatments.

Hereafter, the cosmetic process, kit, and the like according to the present invention will be described in a detailed manner.

[Cosmetic Process]

The cosmetic process according to the present invention for a keratin substance such as skin and lips, comprises the steps of:

applying onto the keratin substance at least one composition comprising at least one coloring agent; and

applying onto the keratin substance, to which the composition has been applied, a microneedle sheet comprising a substrate sheet and a plurality of microneedles on the substrate sheet such that the microneedles are capable of penetrating into the keratin substance.

The step of applying onto a keratin substance at least one composition comprising at least one coloring agent can be performed by using any applicator such as a brush.

It may be preferable that the composition including at least one coloring agent be applied onto the keratin substance in combination of at least one porous sheet such as a woven or non-woven fabric, in order for good contact of the composition with the keratin substance and good coloring on desired site of the keratin substance.

A porous sheet can absorb and maintain the composition comprising at least one coloring agent. Therefore, applying a porous sheet can make it easy for the composition to contact the keratin substance well, and this can contribute to good coloring of the keratin substance.

The type of the above combination is not limited. Thus, a porous sheet may be applied onto the keratin substance before applying the composition on the keratin substance. Alternatively, a porous sheet may be applied onto the keratin substance after applying the composition on the keratin substance.

It is preferable that the step of applying the microneedle sheet onto the keratin substance be performed for a desired area on the keratin substance. For example, it is preferable that the applying site of the microneedle sheet can move on the surface of the keratin substance provided that the microneedles are capable of penetrating into the desired area on the keratin substance.

It is preferable that the microneedle sheet be applied onto the keratin substance without a step of drying the composition on the keratin substance. In other words, it is preferable that the microneedle sheet be applied onto the keratin substance such that the microneedles are capable of penetrating into the keratin substance before the composition is dried or while the keratin substance is wet.

The cosmetic process according to the present invention may further comprise the step of removing the microneedles from the surface of the keratin substance.

After the step of removing the microneedles from the surface of the keratin substance, the composition comprising at least one coloring agent may be removed from the surface of the keratin substance.

It is preferable that the cosmetic process according to the present invention further comprises the step of maintaining or leaving the composition comprising at least one coloring agent on the keratin substance after the step of removing the microneedles from the surface of the keratin substance and before the step of removing the composition from the surface of the keratin substance, because the composition can effectively penetrate into the keratin substance via the holes formed on and in the keratin substance by the microneedles.

The cosmetic process according to the present invention may be intended for cosmetic treatments of a keratin substance such as skin and lips, preferably skin, and more preferably face skin.

The cosmetic process according to the present invention may be used to improve the aesthetic appearance of a keratin substance, for example, by coloring the keratin substance.

The cosmetic process according to the present invention can provide a keratin substance with long lasting and uniform color effects. Therefore, for example, make-up effects provided by the cosmetic process according to the present invention can be maintain for a long period of time even in the face of, for example, sebum, sweat and rain.

However, the cosmetic process according to the present invention does not correspond to skin tattooing, which is semi-permanent coloring, because the skin tattooing is accompanied with pain. Also, the cosmetic process according to the present invention color the superficial layer of a keratin substance such as skin and lips, and therefore, the color provided by the cosmetic process according to the present invention can last, for example, only for a few weeks, which do not correspond to semi-permanent coloring.

Hereafter, the composition, the microneedle sheet and the like used for the cosmetic process according to the present invention, as well as examples of how to perform the cosmetic process according to the present invention, will be explained in a detailed manner.

{Composition}

The composition to be used for the cosmetic process according to the present invention comprises at least one coloring agent.

The composition may be used as a pre-treatment composition for a keratin substance.

(Coloring Agent)

In the present invention, “coloring agent” includes any synthetic or natural, or organic or inorganic, pigments, dyes or lakes, and any other colorants approved by CTFA and the FDA.

In one embodiment, the coloring agent may be selected from dyes, pigments and mixtures thereof.

In the present invention, the coloring agent may be water-soluble or water-dispersible, or oil-soluble or oil-dispersible or with limited solubility in water.

In one embodiment, the coloring agent may be selected from coloring pigments.

The term “coloring pigments” should be understood as meaning white or colored, inorganic or organic particles of any shape which are insoluble and are intended to color or dye the skin or the lips.

The pigments can be white or colored, inorganic and/or organic.

Among the inorganic pigments that may be used, non-limiting mention may be made of titanium dioxide, optionally surface treated, zirconium or cerium oxide, as well as zinc, (black, yellow or red) iron or chromium oxide, manganese violet, ultramarine blue, chromium hydrate and ferric blue, or metal powders, such as aluminum powder or copper powder. The pigments can also be chosen from nanopigments formed of metal oxides, such as titanium dioxide, zinc oxide, iron oxide, zirconium oxide, and cerium oxide, and mixtures thereof. The term “nanopigments” is understood to mean pigments having a mean particle size ranging from 1 nm to 500 nm, such as particle sizes ranging from 10 nm to 100 nm.

Among the organic pigments that may be used, non-limiting mention may be made of carbon black, pigments of D&C type and lakes, such as lakes based on cochineal carmine and on barium, strontium, calcium or aluminum. For example, Red 33 (Disodium 5-amino-4-hydroxy-3-(phenylazo)-naphthalene-2,7-disulfonate) and Red 202 (Calcium bis[2-(3-carboxy-2-hydroxynephthylazo)-5-methylbenzenesulfonate) may be used as the pigment of D&C type.

The organic pigment may also be a diketopyrrolopyrroles (DPP) such as the ones described in EP-A-542669, EP-A-787730, EP-A-787731 and WO-A- 96/08537.

The organic pigment may be selected from bio-pigments such as BioChromaDerm® or BioChromaEyes® provided from Biotic Phocea in France.

Preferably, the coloring pigment may be chosen from metal oxides such as titanium dioxide, zirconium oxide, cerium oxide, zinc oxide, iron oxide, and chromium oxide; manganese violet; Prussian blue; ultramarine blue; chromium hydrate; ferric blue; aluminum powder; copper powder; carbon black; pigments of D&C type; lakes; pearlescent pigments; and mixtures thereof.

The term “pearlescent pigments” should be understood as meaning iridescent particles of any shape, such as particles produced by certain shellfish in their shells or else synthesized.

The pearlescent agents can be chosen from white pearlescent agents, such as mica covered with titanium dioxide or with bismuth oxychloride; colored pearlescent agents, such as titanium oxide-coated mica covered with iron oxide, titanium oxide-coated mica covered with ferric blue or chromium oxide, or titanium oxide-coated mica covered with an organic pigment of the above-mentioned type; and pearlescent agents based on bismuth oxychloride.

In one embodiment, the microneedle may include high refraction index particles, interference particles, reflection particles, and light absorbent particles, in particular non-transparent particles or particles modifying the path of light, as the coloring pigment, in order to prevent reflection. Such particles can allow coloring or dermal-pigmenting of the skin or the lips thanks to the optical properties of the particles.

In another embodiment, the coloring agent may be selected from dyes. The dyes may be selected from natural direct dyes and synthetic direct dyes.

A direct dye means a colored substance which does not require the use of an oxidizing agent in order to develop its color.

In one embodiment, the coloring agent may be selected from natural direct dyes.

The expression “natural direct dye” is understood to mean any dye or dye precursor that is naturally occurring and is produced by extraction (and optionally purification) from a plant matrix or an animal such as an insect, optionally in the presence of natural compounds such as ash or ammonia.

As natural direct dyes, mention may be made of quinone dyes (such as lawsone and juglone), alizarin, purpurin, laccaic acid, carminic acid, kermesic acid, purpurogallin, protocatechaldehyde, indigoids such as indigo, sorghum, isatin, betanin, curcuminoids (such as curcumin), spinulosin, various types of chlorophyll and chlorophyllin, hematoxylin, hematein, brazilein, brazilin, safflower dyes (such as carthamin), flavonoids (such as rutin, quercetin, catechin, epicatechin, morin, apigenidin, and sandalwood), anthocyans (such as apigeninidin and apigenin), carotenoids, tannins, orceins, santalins and cochineal carmine.

It is also possible to use extracts or decoctions containing natural direct dye(s), in particular henna-based extracts, Curcuma longa extract, sorghum leaf-sheath extract, haematoxylon campechianum extract, green tea extract, pine bark extract, cocoa extract, and logwood extract.

It is preferable that the natural direct dye be chosen from the group consisting of curcuminoids, santalins, chlorophyllin, haematoxylin, haematein, brazilein, brazilin, sorghum, laccaic acid, lawsone, juglone, alizarin, purpurin, carminic acid, kermesic acid, purpurogallin, protocatechaldehyde, indigoids, isatin, spinulosin, apigenin, orcein, betanin, flavonoids, anthocyans, and extracts or decoctions containing these compounds.

Alternatively, the natural direct dyes may be preferably chosen, for example, from hydroxylated quinones, indigoids, hydroxyflavones, santalins A and B, isatin and its derivatives, and brasilin and its hydroxylated derivative.

The hydroxylated quinones are preferably benzoquinones, naphthoquinones, and mono- or polyhydroxylated anthraquinones which are optionally substituted with groups such as alkyl, alkoxy, alkenyl, chloro, phenyl, hydroxyalkyl and carboxyl.

The naphthoquinones are preferably lawsone, juglone, flaviolin, naphthazarin, naphthopurpurin, lapachol, plumbagin, chloroplumbagin, droserone, shikonin, 2-hydroxy-3-methyl-1,4-naphthoquinone, 3,5-dihydroxy-1,4-naphthoquinone, 2,5-dihydroxy-1,4-naphthoquinone, 2-methoxy-5-hydroxy-1,4-naphthoquinone and 3-methoxy-5-hydroxy-1,4-naphthoquinone.

The benzoquinones are preferably spinulosin, atromentin, aurentioglyocladin, 2,5-dihydroxy-6-methylbenzoquinone, 2-hydroxy-3-methyl-6-methoxybenzoquinone, 2,5-dihydroxy-3,6-diphenylbenzoquinone, 2,3-dimethyl-5-hydroxy-6-methoxybenzoquinone and 2,5-dihydroxy-6-isopropylbenzoquinone.

The anthraquinones are preferably alizarin, quinizarin, purpurin, carminic acid, chrysophanol, kermesic acid, rhein, aloe emodin, pseudopurpurin, quinizarincarboxylic acid, frangula emodin, 2-methylquinizarin, 1-hydroxyanthraquinone and 2-hydroxyanthraquinone.

The indigoids are preferably indigo, indirubin, isoindigo and Tyrian purple.

The hydroxyflavones are preferably quercetin and morin.

In another embodiment, the coloring agent may be selected from synthetic direct dyes.

The expression “synthetic direct dye” is understood to mean any dye or dye precursor that is produced by chemical synthesis.

The direct dye can be selected from the group consisting of acidic (anionic) direct dyes, basic (cationic) direct dyes, and neutral (nonionic) direct dyes.

Non-limiting examples of synthetic dyes include (nonionic) neutral, anionic (acidic), and cationic (basic) dyes such as azo, methine, carbonyl, azine, nitro(hetero)aryl types or tri(hetero)arylmethane direct dyes, porphyrins and phthalocyanines, alone or as mixtures.

More particularly, the azo dyes comprise an —N═N— functional group, the two nitrogen atoms of which are not simultaneously involved in a ring. However, it is not ruled out for one of the two nitrogen atoms of the —N═N— sequence to be involved in a ring.

The dyes of the family of the methines are more particularly compounds comprising at least one sequence chosen from >C═C< and —N═C<, the two atoms of which are not simultaneously involved in a ring. However, it is specified that one of the nitrogen or carbon atoms of the sequences can be involved in a ring. More particularly, the dyes of this family result from compounds of the following types: true methine (comprising one or more of the above-mentioned —C═C— sequences); azomethine (comprising at least one or more —C═N-sequences) with, for example, the azacarbocyanines and their isomers, the diazacarbocyanines and their isomers, the tetraazacarbocyanines; mono- and diarylmethane; indoamines (or diphenylamines); indophenols; indoanilines.

As regards to the dyes of the family of the carbonyls, mention may be made, for example, of synthetic dyes chosen from acridone, benzoquinone, anthraquinone, naphthoquinone, benzanthrone, anthranthrone, pyranthrone, pyrazolanthrone, pyrimidinoanthrone, flavanthrone, indanthrone, flavone, (iso)violanthrone, isoindolinone, benzimidazolone, isoquinolinone, anthrapyridone, pyrazoloquinazolone, perinone, quinacridone, quinophthalone, naphthalimide, anthrapyrimidine, diketopyrrolopyrrole or coumarin dyes.

As regards to the dyes of the family of the cyclic azines, mention may in particular be made of azine, xanthene, thioxanthene, fluorindine, acridine, (di)oxazine, (di)thiazine or pyronine dyes.

The nitro(hetero)aromatic dyes are more particularly nitrobenzene or nitropyridine direct dyes.

As regards to the dyes of porphyrin or phthalocyanine type, use may be made of cationic or noncationic compounds optionally comprising one or more metals or metal ions, such as, for example, alkali and alkaline earth metals, zinc and silicon.

Mention may be made, as examples of synthetic direct dyes which are particularly suitable, of nitrobenzene dyes, azo, azomethine or methine direct dyes, azacarbocyanines, such as tetraazacarbocyanines (tetraazapentamethines), quinone and in particular anthraquinone, naphthoquinone or benzoquinone direct dyes, or azine, xanthene, triarylmethane, indoamine, phthalocyanine and porphyrin direct dyes, alone or as mixtures. More preferably still, these synthetic direct dyes are chosen from nitrobenzene dyes, azo, azomethine or methine direct dyes and tetraazacarbocyanines (tetraazapentamethines); alone or as mixtures.

Mention may be made, among the azo, azomethine, methine or tetraazapentamethine direct dyes which can be used according to the invention, of the cationic dyes described in Patent Applications WO 95/15144, WO 95/01772 and EP 714 954; FR 2 189 006, FR 2 285 851, FR-2 140 205, EP 1 378 544 and EP 1 674 073.

Thus, mention may very particularly be made of the cationic direct dyes corresponding to the following formulae:

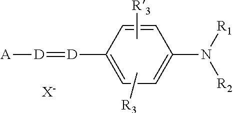

in which:

D represents a nitrogen atom or the —CH group,

R₁ and R₂, which are identical or different, represent a hydrogen atom; a C₁-C₄ alkyl radical which can be substituted by a —CN, —OH or —NH₂ radical or can form, with a carbon atom of the benzene ring, an optionally oxygen-comprising or nitrogen-comprising heterocycle which can be substituted by one or more C₁-C₄ alkyl radicals; or a 4′-aminophenyl radical,

R₃ and R′₃, which are identical or different, represent a hydrogen atom, a halogen atom chosen from chlorine, bromine, iodine and fluorine, a cyano radical, a C₁-C₄ alkyl radical, a C₁-C₄ alkoxy radical or an acetyloxy radical,

X⁻ represents an anion, preferably chosen from chloride, methyl sulphate and acetate,

A represents a group chosen from the following structures:

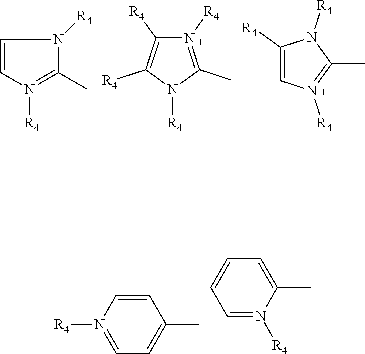

in which R₄ represents a C₁-C₄ alkyl radical which can be substituted by a hydroxyl radical;

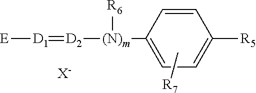

in which:

R₅ represents a hydrogen atom, a C₁-C₄ alkoxy radical or a halogen atom, such as bromine, chlorine, iodine or fluorine,

R₆ represents a hydrogen atom or a C₁-C₄ alkyl radical or forms, with a carbon atom in the benzene ring, a heterocycle which optionally comprises oxygen and/or is optionally substituted by one or more C₁-C₄ alkyl groups,

R₇ represents a hydrogen atom or a halogen atom, such as bromine, chlorine, iodine or fluorine,

D₁ and D₂, which are identical or different, represent a nitrogen atom or the —CH group, m=0 or 1,

X⁻ represents a cosmetically acceptable anion preferably chosen from chloride, methyl sulphate and acetate,

E represents a group chosen from the following structures:

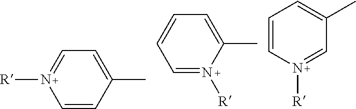

in which R′ represents a C₁-C₄ alkyl radical;

when m=0 and when D₁ represents a nitrogen atom, then E can also denote a group with the following structure:

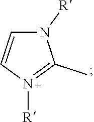

in which R′ represents a C₁-C₄ alkyl radical.

The synthetic direct dye may be selected from fluorescent dyes. Two or more types of fluorescent dyes may be used in combination.

The use of some fluorescent dyes may make it possible to obtain, on dark hair, colors which are more visible than with conventional hydrophilic or hydrophobic direct dyes. Furthermore, these fluorescent dyes, when applied to dark hair, may also make it possible to lighten the hair without damaging it.

As used herein, the term “fluorescent dyes” is understood to mean fluorescent compounds and optical brighteners. In at least one embodiment, the fluorescent dye is soluble in the skin or the lips.

Fluorescent dyes are fluorescent compounds which absorb visible radiation, for example, wavelengths ranging from 400 to 800 nm, and which are capable of re-emitting light in the visible region at a higher wavelength.

According to one embodiment, the fluorescent dyes useful for the present invention re-emit orange-colored fluorescent light. They exhibit, for instance, a maximum re-emission wavelength ranging from 500 to 700 nm.

Non-limiting examples of fluorescent dyes include compounds known in the art, for example, those described in Ullmann's Encyclopedia of Industrial Chemistry, Release 2004, 7th edition, “Fluorescent Dyes” chapter.

The optical brighteners of the present disclosure, also known under the name of “brighteners”, or “fluorescent brighteners”, or “fluorescent brightening agents” or “FWA”, or “fluorescent whitening agents”, or “whiteners”, or “fluorescent whiteners”, are colorless transparent compounds as they do not absorb in visible light but only in ultraviolet light (wavelengths ranging from 200 to 400 nanometers) and convert the energy absorbed into fluorescent light of higher wavelength emitted in the visible part of the spectrum, generally in the blue and/or green, that is to say in wavelengths ranging from 400 to 550 nanometers.

Optical brighteners are known in the art, for example, they are described in Ullmann's Encyclopedia of Industrial Chemistry (2002), “Optical Brighteners” and Kirk-Othmer Encyclopedia of Chemical Technology (1995): “Fluorescent Whitening Agents”.

The fluorescent dyes which can be used in the present invention include compounds known from the art, for example, those described in French Patent No. 2 830 189.

Soluble fluorescent compounds that may especially be mentioned include those belonging to the following families: naphthalimides, coumarins, xanthenes and in particular xanthenodiquinolizines and azaxanthenes; naphtholactams; azlactones; oxazines; thiazines; dioxazines; azo compounds; azomethines; methines; pyrazines; stilbenes; ketopyrroles; and pyrenes.

If present, the fluorescent dyes are preferred, more particularly, those re-emitting orange-colored fluorescent light.

In terms of ionic nature, the direct dye may be selected from the group consisting of acidic direct dyes, basic direct dyes and neutral direct dyes, which covers all possible types of direct dyes, such as so-called nitro dyes and HC dyes. Acidic direct dyes have an anionic moiety in their chemical structure. Basic direct dyes have a cationic moiety in their chemical structure. Neutral direct dyes are nonionic.

In a preferred embodiment, the coloring agent may be selected from dyes, preferably natural direct dyes, and more preferably coloring plant extracts.

The coloring plant extracts here mean plant extracts which are capable of coloring. As coloring plant extracts, mention may be made of: carrot extract, gardena florida fruit extract, saffron extract, orange extract, and tomato extract, in addition to those explained above for the natural direct dye.

In particular, the coloring agent may be selected from the following compounds and mixtures thereof.

| Color | Commercial Name | Chemical Name |
|---|---|---|
| Black | Sunpuro Black Iron Oxide C33-7001 | Iron Oxide |
| Black | Unipure Triple Black Lc990 | Iron Oxide |
| Black | Unipure Black Lc902 | Carbon Black |
| Black | Wd-Cb2 | Carbon Black |
| Black | Black Nf | Iron Oxide |
| Blue | Suncroma Iron Blue C38-5410 | Ferric |
| | | Ferrocyanide |
| Blue | Unipure Blue Lc 621 | Blue 1 Lake |
| Blue | Unipure Blue Lc 686 | Ultramarine Blue |
| Blue | Unipure Blue Lc 520 | Carmine |
| Bordeaux | Suncroma D&C Red 34 Ca Lake C24- | Red 34 Lake |
| | 012 | |
| Green | Unipure Green Lc 788 | Chromium Oxide |
| | | Green |
| Orange | Suncroma Fd&C Yellow 6 Al Lake | Yellow 6 Lake |
| | C70-5270 | |
| Pink | Suncroma D&C Red 28 Al Lake C14- | Red 28 Lake |
| | 6623 | |
| Pink | Dell Red C-14-6634/Suncroma D&C | Red 22 Lake |
| | Red 22 Al Lake C14-6634 | |
| Pink | Unipure Pink Lc 589 | Ultramarines |
| Red | Unipure Red Lc 3079 Or | Red 7 |
| Red | Carmin Covalac W 3508/Cosmetic | Carmine |
| | Carmine 09350/Carmin Rmam 52% | |
| Red | Unipure Red Lc 320 Carmihpc 54% | Carmine |
| Red | Suncroma D&C Red 6 Ba Lake C-19-012 | Red 6 Lake |
| Red | Suncroma D&C Red 6 Na Salt C19-6619 | Red 6 Na Salt |
| Red | Suncroma D&C Red 7 Ca Lake C-19-003 | Red 7 Lake |
| Red | Suncroma D&C Red 7 Ca Lake C19-011 | Red 7 Lake |
| Red | Suncroma D&C Red 7 Ca Lake C19-025 | Red 7 Lake |
| Red | Suncroma D&C Red 30al Lake C37-038 | Red 30 Lake |
| Red | D & C Red 33 K7057 | Red 33 |
| Red | Suncroma D&C Red 33 Al Lake C17- | Red 33 Lake |
| | 6444 | |
| Red | Suncroma Fd&C Red 40 Al Lake C37- | Red 40 Lake |
| | 6340 | |
| Red | Sunpuro Red Iron Oxide C33-8001 | Iron Oxide |
| Red | Unipure Red Lc 383 | Iron Oxide |
| Violet | Unipure Violet Lc 581 | Manganese Violet |
| Violet | Unipure Violet Lc 587 | Ultramarine |
| | | Violet |
| Violet | Unipure Blue Lc 520 | Carmine |
| White | Hombitan Anatase Ff Pharma | Titanium Dioxide |
| White | HOMBITAN AFDC300 | Titanium Dioxide |
| White | Tipaque Pf-671 | Titanium Dioxide |
| White | Tipaque Pfc407 | Titanium Dioxide |
| White | Sachtleben Rc 402 | Titanium Dioxide |
| White | KRONOS 1171 | Titanium Dioxide |
| White | Tipaque Cr-50 | Titanium Dioxide |
| Yellow | Suncroma Fd&C Yel 5 Al Lk C69-4424 | Yellow 5 Lake |
| Yellow | Fd&C Yel 5 Al W001 | Yellow 5 Lake |
| Yellow | Suncroma Fd&C Yel 5 Al Lk C69002 | Yellow 5 Lake |
| Yellow | Unipure Yellow Lc124 | Yellow 10 Lake |
| Yellow | Sunpuro Yellow Iron Oxide C33-9001 | Iron Oxide |
| Yellow | Suncroma Fd&C Yellow 6 Al Lake C70- | Yellow 6 Lake |
| | 5270 | |

The amount of the coloring agent in the microneedle of the microneedle sheet according to the present invention is not limited.

The amount of the coloring agent in the composition to be used for the cosmetic process according to the present invention may be 0.001% by weight or more, preferably 0.01% by weight or more, and more preferably 0.1% by weight or more, relative to the total weight of the composition.

The amount of the coloring agent in the composition to be used for the cosmetic process according to the present invention may be 20% by weight or less, preferably 15% by weight or less, and more preferably 10% by weight or less, relative to the total weight of the composition.

Thus, the amount of the coloring agent in the composition to be used for the cosmetic process according to the present invention may range from 0.001% to 20% by weight, preferably from 0.01% to 15% by weight, and more preferably from 0.1% to 10% by weight relative to the total weight of the composition.

(Vehicle)

The composition to be used for the cosmetic process according to the present invention may comprise at least one vehicle.

The vehicle in the composition to be used for the cosmetic process according to the present invention is not limited.

For example, the vehicle may be selected from hydrophilic media and hydrophobic media.

The hydrophilic media may be alcohols: in particular monovalent alcohols such as ethyl alcohol, isopropyl alcohol, benzyl alcohol, and phenylethyl alcohol; diols such as ethylene glycol, propylene glycol, and butylene glycol; other polyols such as glycerol, sugar, and sugar alcohols; and ethers such as ethylene glycol monomethyl, monoethyl, and monobutyl ethers, propylene glycol monomethyl, monoethyl, and monobutyl ethers, and butylene glycol monomethyl, monoethyl, and monobutyl ethers.

The hydrophobic media may be selected from oils.

It may be preferable that the composition to be used for the cosmetic process according to the present invention comprise water.

The amount of the vehicle in the composition to be used for the present invention may be 50% by weight or more, preferably 55% by weight or more, and more preferably 60% by weight or more, relative to the total weight of the composition.

The amount of the vehicle in the composition to be used for the present invention may be 99% by weight or less, preferably 95% by weight or less, and more preferably 90% by weight or less, relative to the total weight of the composition.

The amount of vehicle in the composition to be used for the present invention may range from 50% to 99% by weight, preferably from 55% to 95% by weight, and more preferably from 60% to 90% by weight, relative to the total weight of the composition.

(Optional Ingredients)

The composition to be used for the cosmetic process according to the present invention may comprise, in addition to the aforementioned ingredients, ingredients typically employed in cosmetics, specifically, surfactants or emulsifiers, hydrophilic or lipophilic thickeners, natural extracts derived from animals or vegetables, waxes, and the like, within a range which does not impair the effects of the present invention.

The composition to be used for the cosmetic process according to the present invention may comprise the above optional additive(s) in an amount of from 0.01% to 50% by weight, preferably from 0.05% to 30% by weight, and more preferably from 0.1% to 10% by weight, relative to the total weight of the composition.

(Preparation)

The composition to be used for the present invention can be prepared by, for example, mixing coloring agent(s) and vehicle(s), as essential ingredients, and optional ingredient(s), if necessary, as explained above.

The method and means to mix the above essential and optional ingredients are not limited. Any conventional method and means can be used to mix the above essential and optional ingredients to prepare the composition to be used for the present invention.

{Microneedle Sheet}

The microneedle sheet to be used for the process according to the present invention comprises a substrate sheet and a plurality of microneedles on a substrate sheet.

The microneedle sheet to be used for the present invention may be a cosmetic device, preferably a cosmetic device for a keratin substance, and more preferably a cosmetic device for skin, in particular face skin, as well as lips.

(Microneedles)

The microneedle sheet to be used for the present invention comprises a plurality of microneedles.

The microneedles are present on the surface of a substrate sheet. The microneedles may be present on 50% or more, preferably 70% or more, and more preferably 90% or more of the surface of the substrate sheet.

It is preferable that the microneedles be present on one of the surfaces of a substrate sheet.

It is preferable that the microneedles of the microneedle sheet to be used for the present invention be designed to penetrate or enter into the stratum corneum of skin, in particular face skin, as well as lips.

A microneedle can be any suitable size and shape to puncture the stratum corneum. It may be preferable that the microneedles be designed to pierce and cross the stratum corneum. The microneedles may be capable of creating openings or passages in the stratum corneum.

If necessary, the height of the microneedles may be adjusted so as to allow penetration to the epidermis and/or dermis of the skin, preferably to the epidermis, and more preferably to any one of the layers in the epidermis.

The shape of the microneedles is not limited as long as the shape is a “needle”. It will be apparent to those skilled in the art that the microneedles for the present invention can take any reasonable shape, including, but not limited to, pyramids, cones, rods and/or pillars. As such, the microneedles may have the same diameter at the tip as at the base or may taper in diameter in the direction from the base to the tip.

For example, the shape of the microneedle may be in the form of a triangular pyramid, a square pyramid or a pentagonal pyramid. Alternatively, the microneedle may be in the form of a cylinder preferably with a tip which may be formed by diagonally cutting the cylinder. The cross section of the microneedle may take any geometric form including, circular, triangular, square, rectangular, polyhedral, regular or irregular forms, and the like. In an embodiment, a group of microneedles may take the form of hollow microcapillaries. However, for the present invention, solid (non-hollow) microneedles may be preferable.

Thus, reference is made to “microneedles” as a type of microprotrusion or microprojection which is being employed. It will be understood by persons skilled in the art that in many cases the same inventive principles apply to the use of other microprotrusions or microprojections to penetrate the skin. Other microprotrusions or microprojections may include, for example, microblades as described in U.S. Pat. No. 6,219,574 and Canadian Patent Application No. 2,226,718, and edged microneedles as described in U.S. Pat. No. 6,652,478.

The height or length of the microneedle of the microneedle sheet to be used for the present invention may be from 10 to 500 microns, preferably from 30 to 300 microns, and more preferably from 50 to 150 microns.

According to one embodiment of the present invention, the microneedle is in the form of a cone.

The cone may comprise a distal end such as a tip, and a base. The shape of the base may be a circle or oval.

The height or length of the cone of the microneedle of the microneedle sheet to be used for the present invention may be from 10 to 500 microns, preferably from 30 to 300 microns, and more preferably from 50 to 150 microns.

The base of the cone of the microneedle of the microneedle sheet to be used for the present invention may have a diameter or width of from 10 to 500 microns, preferably from 10 to 300 microns, and more preferably from 10 to 100 microns. If the base of the cone of the microneedle of the microneedle sheet to be used for the present invention is in the shape of an oval or ellipse, the length of the major axis or width of the oval may be from 10 to 500 microns, preferably from 10 to 300 microns, and more preferably from 10 to 100 microns.

The microneedle may have an aspect ratio (length/width at base) of at least about 3:1, at least about 2:1, or at least about 1:1. The ratio of (the height of the cone)/(the diameter of the base of the cone) of the microneedle may be 1 or more, preferably 1.5 or more, and more preferably 2.0 or more.

According to another embodiment of the present invention, the microneedle is in the form of a pyramid, such as a triangular pyramid and a square pyramid.

The pyramid may comprise a distal end such as a tip, and a base. The shape of the base may be a triangle or a square.

The height or length of the pyramid of the microneedle of the microneedle sheet to be used for the present invention may be from 10 to 500 microns, preferably from 30 to 300 microns, and more preferably from 50 to 150 microns.

The base of the pyramid of the microneedle of the microneedle sheet to be used for the present invention may have a width of from 10 to 500 microns, preferably from 10 to 300 microns, and more preferably from 10 to 100 microns. If the base of the pyramid of the microneedle of the microneedle sheet to be used for the present invention is in the shape of a triangle or square, the length of the side or width of the triangle or square may be from 10 to 500 microns, preferably from 10 to 300 microns, and more preferably from 10 to 100 microns.

The microneedle may have an aspect ratio (length/width at base) of at least about 3:1, at least about 2:1, or at least about 1:1. The ratio of (the height of the pyramid)/(the width of the base of the pyramid) of the microneedle may be 1 or more, preferably 1.5 or more, and more preferably 2.0 or more.

It may be preferable that the microneedle be capable of penetrating into a keratin substance, such as skin and lips, to a depth of 200 microns or less, preferably 180 microns or less, and more preferably 160 microns or less.

A very narrow pitch of microneedles may not provide the microneedles with sufficient surface area so that the microneedles may not penetrate inside the skin. On the other hand, a too wide pitch may also cause issue that individual microneedle may not get enough pressure to penetrate. Thus, it may be preferable that a pitch of microneedle arrays be from 400 to 700 microns, and more preferably from 400 to 500 microns.

The number of the microneedles and the pitch between the microneedles depend on some factors such as desired color pattern and width of a microneedle. For example, if a color pattern with a 0.5 micron width is desired, the use of a microneedle sheet having a microneedle array (2×2) with a 0.5 mm pitch may be the most appropriate.

In one embodiment, the density of microneedles may be from 100 to 2000 microneedles/cm², preferably 200 to 1000 microneedles/cm², and even more preferably 200 to 500 microneedles/cm².

It is preferable that the microneedles are not dissolvable.

The “not dissolvable” microneedle is meant that the microneedle cannot break down or disintegrated inside a keratin substance such as skin and lips by, for example, a natural moisturizing factor or external moisture.

It is more preferable that the microneedles are not water-soluble.

The microneedles may be made from organic or inorganic materials. As examples of the organic materials, mention may be made of synthetic polymers including polyethylene, polypropylene and photoresists, biodegradable plastics, cellulose derivatives and mixtures thereof. As examples of the inorganic materials, mention may be made of metals including stainless steel, non-metals such as silicon, glass, and ceramics, and mixtures thereof. Also, any other materials which is implantable in skin or body may be used.

It is preferable that the microneedles be sterile or sterilized before use. It is more preferable that the microneedles be used for only once, and therefore be disposable.

It is preferable that the microneedles be made from inorganic materials, more preferably metals or non-metals, and even more preferably silicon (in particular, single crystal silicon).

It is also preferable that the microneedles be solid (non-hollow).

The microneedles themselves may have any color. However, it may be preferable that the microneedles do not include any coloring agent in order to color a keratin substance.

(Substrate Sheet)

The microneedle sheet according to the present invention comprises a substrate sheet on which the microneedles are present or placed.

The substrate sheet and the microneedles may be separated or integrated.

For example, the substrate sheet and the microneedles may comprise at least one common material. Thus, in one embodiment, the substrate sheet and the microneedles can be a single element comprising at least one common material. Preferably, the single element can be prepared by using the same material.

On the other hand, the substrate sheet may be different or distinct from the microneedles. For example, the substrate sheet and the microneedles may be made from different materials. In this case, the substrate sheet may be, for example, chosen from masks, wipes, patches, and in general all types of porous substrate sheets. Preferably, these substrate sheets have an oblong structure, namely with a thickness smaller than the dimensions of the plane in which they are defined.

The substrate sheet may be cut so as to be in the form of a patch, a disc, a mask, a towel, a glove, a precut roll, or any other form suitable for a cosmetic use.

(Preparation)

There is no limitation regarding how to prepare the microneedle sheet to be used for the present invention. It is possible to prepare the microneedle sheet to be used for the present invention based on conventional technology such as molding, 3D printing, metal processing, and the like.

The shape of the microneedle sheet to be used for the present invention is not limited, and it may be any shape such as the shape of the lips or a shape suitable for application under the eyes, depending on the application target of the microneedle sheet.

{Device}

In the cosmetic process according to the present invention, the microneedle sheet is applied onto a keratin substance such as skin and lips such that the microneedles are capable of penetrating into the keratin substance.

It is preferable that the microneedles be capable of penetrating into the keratin substrate with repetitions.

It is not limited how to perform repeating penetrations of microneedles into a keratin substance. For example, the repeating penetrations may be performed by tapping the back surface of the substrate sheet of the microneedle sheet with fingers several times such that the microneedles on the front surface of the substrate sheet can repeatedly penetrate into the keratin substance.

Furthermore, it is preferable to perform the repeating penetrations of microneedles by using vibration toward a keratin substance such as skin and lips.

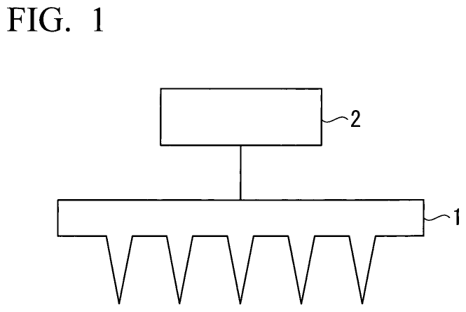

For example, as shown in FIG. 1, a microneedle sheet 1 can be connected to an oscillator 2 which can produce vibration toward the surface of a keratin substance which corresponds to the direction of up and down in FIG. 1. The type of the oscillator 2 is not limited as long as it can produce vibration. For example, the oscillator 2 may be constituted by a piezo electric element. Of course, any other actuator which can repeatedly move, preferably vibrate, toward a keratin substance can be used instead of the oscillator 2.

In FIG. 1, the microneedle sheet 1 can move up and down based on the vibration caused by the oscillator 2, and therefore, the microneedles on the microneedle sheet 1 can repeatedly penetrate into a keratin substance such as skin and lips.

It is preferable that the oscillator 2 be linked with a head which can function as a receptor for the microneedle sheet 1, and that the substrate sheet of the microneedle sheet 1 is attached to the head. Thus, the vibration caused by the oscillator 2 can be conveyed to the head. Then, the microneedle sheet 1 can also vibrate via the head.

It is preferable to use a device in order to vibrate the microneedle sheet 1. For example, the device may comprise the oscillator 2 and the head which is linked with the oscillator 2 as well as a battery, such as a dry cell, which can drive the oscillator 2 and a control means to control the frequency of the vibration caused by the oscillator 2. As example, a device disclosed in WO 2011/115602, the content of which is incorporated herein by reference, may be used as the above device.

When using the above device, the microneedle sheet 1 can be mounted on the head of the device, and can be applied onto a keratin substance such as skin and lips such that the microneedles of the microneedle sheet 1 can repeatedly penetrate into the keratin substance.

The vibrating or tapping frequency is not limited. For example, the frequency may be from 1000 to 10000 per minute, preferably from 1000 to 8000 per minute, and more preferably from 3000 to 6000 per minute.

It may be preferable to control the pressure to be used for penetrating the microneedles into a keratin substance. The pressure can depend on some factors such as the type of microneedles as well as the type of coloring.

The tapping or vibrating penetration of microneedles enables continuous and stable skin puncture, and can reduce the force required to drive the microneedle into the keratin substance. It may further provide massage effects to the treated area of the keratin substance, allowing a better diffusion or dispersion of the coloring agent into the keratin substance.

### Examples of How to Perform the Cosmetic Process

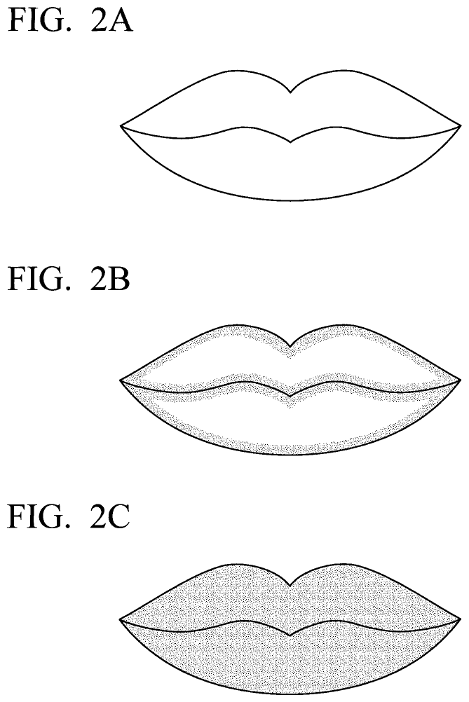

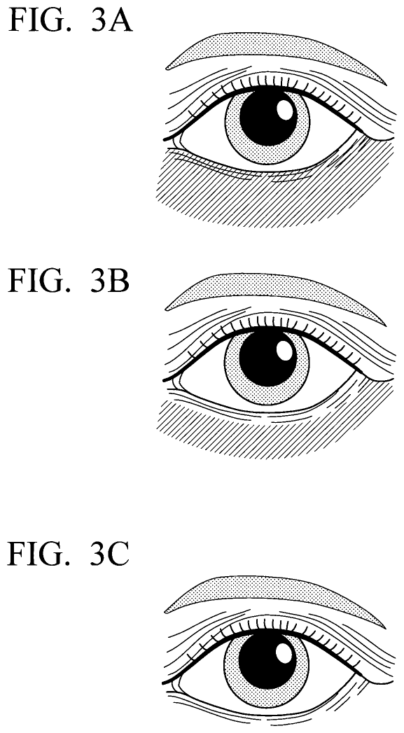

FIGS. 2 and 3 show some examples of how to perform the cosmetic process according to the present invention.

FIG. 2A-2C illustrate some ways of long lasting lip coloring.

FIG. 2A shows a human lip. The breadth of each of the lips along with the longitudinal direction of the figure is about 15 mm.

FIG. 2B shows an example of lip coloring in which, first, a composition including at least one coloring agent is applied, and then, a microneedle sheet with an array of microneedles (2×2) is applied along with the 0.4 mm width of the edges of the lips. As shown in FIG. 2B, the edges of the lips can be colored.

FIG. 2C shows an another example of lip coloring in which, first, a composition including at least one coloring agent is applied, and then, a microneedle sheet with an array of microneedles (10×10) is applied on the whole of the lips. As shown in FIG. 2C, the whole of the lips can be colored.

FIG. 3A-3C illustrate some ways of long lasting coloring under eye.

FIG. 3A shows a human eye. The shaded area is dark and is herein called “dark eye circle” which has a breadth along with the longitudinal direction of the figure of about 15 mm.

FIG. 3B shows an example of coloring under eye in which, first, a composition including at least one coloring agent (with normal skin color) is applied, and then, a microneedle sheet with an array of microneedles (2×2) is applied along with the 0.4 mm width of the edges of the dark eye circle. As shown in FIG. 3B, the edges of the dark eye circle are covered by the coloring agent, and therefore, it looks that the area of the dark eye circle is reduced.

FIG. 3C shows an another example of coloring under eye in which, first, a composition including at least one coloring agent (with normal skin color) is applied, and then, a microneedle sheet with an array of microneedles (10×10) is applied on the whole of the dark eye circle. As shown in FIG. 3C, the whole of the dark eye circle is covered by the coloring agent, and therefore, it looks that no dark eye circle is present.

[Kit, Process, and Use]

The present invention also relates to a kit for coloring a keratin substance, such as skin and lips, comprising:

a microneedle sheet comprising a substrate sheet and a plurality of microneedles on the substrate sheet;

a device for applying the microneedle sheet onto the keratin substance such that the microneedles are capable of penetrating into the keratin substance; and a composition comprising at least one coloring agent.

The above explanations for the microneedle sheet and the composition for the cosmetic process according to the present invention can also apply to those in the kit according to the present invention. In addition, the explanations regarding the above device which may be used for the cosmetic process according to the present invention can also apply to that in the kit according to the present invention.

The composition comprising at least one coloring agent may be contained in any container such as a vessel which is commonly used for cosmetic purposes.

The kit can preferably be used for performing a cosmetic process according to the present invention.

It is preferable that the device in the kit according to the present invention be capable of applying the microneedle sheet with repetitions. For example, the device may comprise at least one element which is capable of vibrating or tapping the microneedle sheet. This element may correspond to the back surface of the substrate sheet if tapping with fingers is used for repeating the penetration of microneedles into a keratin substance, and to the oscillator 2 explained above if vibration is used for repeating the penetration of microneedles into a keratin substance.

The present invention also relates to a use of at least one composition comprising at least one coloring agent in combination with a microneedle sheet comprising a substrate sheet and a plurality of microneedles on the substrate sheet, in order to provide a keratin substance such as skin or lips with long-lasting and uniform color.

Specifically, the use according to the present invention may comprise the first application onto the keratin substance at least one composition comprising at least one coloring agent; and the second application onto the keratin substance, to which the composition has been applied, a microneedle sheet comprising a substrate sheet and a plurality of microneedles on the substrate sheet such that the microneedles are capable of penetrating into the keratin substance, in order to provide a keratin substance such as skin or lips with long-lasting and uniform color.

It is preferable that the microneedles be capable of penetrating into the keratin substrate with repetitions. The repetitions may be based on the vibrating or tapping of the microneedle sheet.

Another aspect of the use of according to the present invention may be a composition comprising at least one coloring agent, for use in long-lasting and uniform coloring of a keratin substance such as skin and lips in combination with a microneedle sheet comprising a substrate sheet and a plurality of microneedles on the substrate sheet wherein the microneedles are capable of penetrating into the keratin substance.

According to the above kit and use according to the present invention, the microneedles of a microneedle sheet can deliver the coloring agent into a keratin substance such as skin and lips provide the keratin substance with long lasting coloring. Also, the above kit and use according to the present invention can provide a keratin substance such as skin and lips with uniform coloring.

### EXAMPLES

The present invention will be described in a more detailed manner by way of examples.

However, these examples should not be construed as limiting the scope of the present invention. The examples below are presented as non-limiting illustrations in the field of the present invention.

### Example 1 (Ex-Vivo Coloring Test 1)

(Microneedle Device)

A square microneedle sheet with 7×7 microneedles on a substrate sheet was used. Each microneedle had a shape of a pyramid with a length or height of 80 μm and a base diameter of m. The microneedles were made from single crystal silicon.

The square microneedle sheet was mounted on a head (first end) of a device as shown in WO 2011/115602, wherein the head can vibrate along with the axis of the device, to prepare a microneedle device which can apply the microneedle sheet onto skin with vibration action toward the skin such that the microneedles can repeatedly penetrate into the keratin substance.

(Composition)

An aqueous solution of 0.1% by weight of Red 33 was used.

(Evaluation)

First, a human cadaver skin with a shape of 1.5 cm×1.5 cm square was provided.

Second, the above composition in the form of an aqueous solution with a concentration of 0.1% by weight of Red 33 was applied onto a site of the skin.

Third, the microneedle device was used to apply the microneedle sheet onto the above skin site such that the microneedles repeatedly penetrated into the above skin site for 5 minutes with a vibration of a frequency of 5000 times per minute.

After the application, the above composition was left and maintained on the above skin site for 15 minutes. A photograph of the surface of the above skin site was taken just after wiping off the composition from the above skin site with wet and dry cotton swabs. After 24 hours, tape stripping was performed 20 times on the surface of the above skin site in order to remove the stratum corneum thereof, and a photograph of the surface of the above skin site was taken.

On the other hand, a tape stripping was performed for another site of the skin, as a control, wherein the above composition was not applied (without coloring treatment), and a photograph of the surface of this skin site was taken.

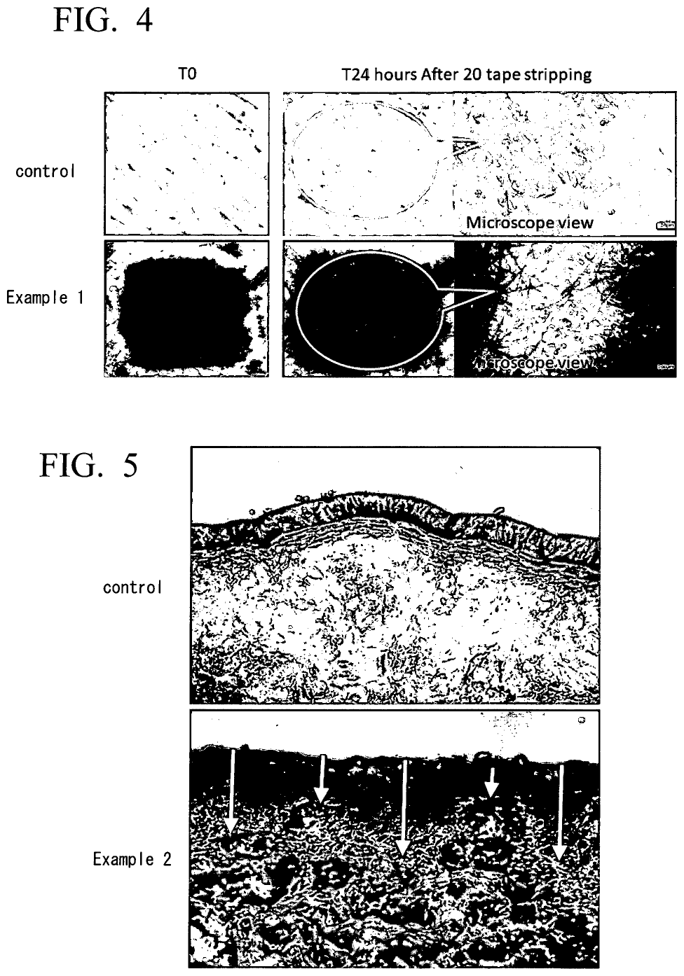

The above photographs are shown in FIG. 4.

As shown in “control” in FIG. 4, just after skin wiping off (cf. TO), slight red color was observed on the skin site without the coloring treatment. However, the color was no more observed after removal of the stratum corneum with the tape stripping as shown in the microscope view of “T24 hours After 20 tape stripping”.

In contrast, as shown in “Example 1” in FIG. 4, the skin site with the coloring treatment was successfully colored with red on the whole treatment area. Moreover, as shown in the microscope view of “T24 hours After 20 tape stripping”, the red color was still clear and uniformly observed even after removal of the stratum corneum. These results demonstrate long-lasting coloring efficacy and uniform coloring to epidermis in Example 1.

### Example 2 (Ex-Vivo Coloring Test 2)

(Microneedle Device)

A square microneedle sheet with 7×7 microneedles on a substrate sheet was used. Each microneedle had a shape of a pyramid with a length or height of 80 m and a base diameter of 40 μm. The microneedles were made from single crystal silicon.

The square microneedle sheet was mounted on a head (first end) of a device as shown in WO 2011/115602, wherein the head can vibrate along with the axis of the device, to prepare a microneedle device which can apply the microneedle sheet onto skin with vibration action toward the skin such that the microneedles can repeatedly penetrate into the keratin substance.

(Composition)

An aqueous solution of 0.1% by weight of Red 33 was used.

(Evaluation)

First, a human cadaver skin with a shape of 1.5 cm×1.5 cm square was provided.

Second, the above composition in the form of an aqueous solution with a concentration of 0.1% by weight of Red 33 was applied onto a site of the skin.

Third, the microneedle device was used to apply the microneedle sheet onto the above skin site such that the microneedles penetrated into the above skin site for 5 minutes with a vibration of a frequency of 5000 times per minute.

After the application, the above composition was left and maintained on the above skin site for 15 minutes. Then, the skin was dissected, put into O.C.T. (Optimal Cutting Temperature) Compound (from Sakura Finetek) and frozen at from −20° C. to −80° C. A cross-sectional sample of the skin with a thickness of 5 to 15 μm was prepared with a Cryostat. The cross-sectional sample was observed with a microscope, and a photograph thereof was taken, in order to evaluate the penetration depth of Red 33 along with the cross-sectional direction.

A cross-sectional sample of the skin onto which the composition was not applied, as a control, was also prepared and observed with a microscope, and a photograph thereof was taken.

The above photographs are shown in FIG. 5.

As shown in “control” in FIG. 5, without the coloring treatment, Red 33 could not penetrate into the skin. In contrast, as shown in “Example 2” of FIG. 5, the skin site with the coloring treatment was successfully colored with red on the whole treatment area, and the penetration depth of Red 33 was through the stratum corneum to the epidermis (cf. arrows). These results also demonstrate long-lasting coloring efficacy and uniform coloring to epidermis in Example 2.

1. A cosmetic process for a keratin substance such as skin and lips, comprising the steps of:
applying onto the keratin substance at least one composition comprising at least one coloring agent; and
applying onto the keratin substance, to which the composition has been applied, a microneedle sheet comprising a substrate sheet and a plurality of microneedles on the substrate sheet such that the microneedles are capable of penetrating into the keratin substance.

2. The cosmetic process according to claim 1, wherein the microneedle has a height of from 10 to 500 microns.

3. The cosmetic process according to claim 1, wherein the microneedle is in the shape of a pyramid.

4. The cosmetic process according to claim 3, wherein the base of the microneedle has a width of from 10 to 500 microns.

5. The cosmetic process according to claim 3, wherein the ratio of (the height of the pyramid)/(the width of the base of the pyramid) of the microneedle is 1 or more.

6. The cosmetic process according to claim 1, wherein the microneedles are made from inorganic materials.

7. The cosmetic process according to claim 1, wherein the microneedles are capable of penetrating into the keratin substrate with repetitions.

8. The cosmetic process according to claim 7, wherein the repetitions are based on the vibrating or tapping of the microneedle sheet.

9. The cosmetic process according to claim 1, wherein the coloring agent is selected from dyes, pigments and mixtures thereof.

10. The cosmetic process according to claim 1, wherein the amount of the coloring agent is from 0.001% to 20% by weight, relative to the total weight of the composition.

11. The cosmetic process according to claim 1, wherein the microneedle sheet is applied onto the keratin substance without a step of drying the composition on the keratin substance.

12. A kit for coloring a keratin substance, such as skin and lips, comprising:
a microneedle sheet comprising a substrate sheet and a plurality of microneedles on the substrate sheet;
a device for applying the microneedle sheet onto the keratin substance such that the microneedles are capable of penetrating into the keratin substance; and
a composition comprising at least one coloring agent.

13. The kit according to claim 12, wherein the device is capable of applying the microneedle sheet with repetitions.

14. The kit according to claim 13, wherein the device comprises at least one element which is capable of vibrating or tapping the microneedle sheet.

15. A use of at least one composition comprising at least one coloring agent in combination with a microneedle sheet comprising a substrate sheet and a plurality of microneedles on the substrate sheet, in order to provide a keratin substance such as skin and lips with long-lasting and uniform color.
